(19) United States
(12) Patent Application Publication
(10) Pub. No.: US 2023/0321606 A1
(43) Pub. Date: Oct. 12, 2023

(54) HIGHLY STABLE LIPID BILAYER (HSLB) WITH BIOPOLYMER SCAFFOLD AS CYTOSKELETON AND USE THEREOF

(71) Applicants: Xiaolong Luo, Laurel, MD (US); Piao Hu, Carry, NC (US)

(72) Inventors: Xiaolong Luo, Laurel, MD (US); Piao Hu, Carry, NC (US)

(21) Appl. No.: 17/714,634

(22) Filed: Apr. 6, 2022

(51) Int. Cl. B01D 67/00; A61K 9/127; A61K 47/36

(52) U.S. Cl. CPC B01D 67/0002; A61K 9/1277; A61K 47/36; A61K 9/1273

(57) ABSTRACT

This invention provides a long-lasting artificial cell membrane with a prefabricated, freestanding biopolymer hydrogel as the cytoskeleton that is partially tethered to and supports lipid bilayer for high stability. The highly stable lipid bilayer has unrestricted fluidic, optical and electrical accesses to both sides of the lipid bilayer, which has significant impact on fundamental biological studies and advanced pharmaceutical industries.

### CROSS-REFERENCES TO RELATED APPLICATIONS

This application claims benefit of priority to U.S. Provisional Patent Application No. 63/174,662, entitled “HIGHLY STABLE LIPID BILAYER (HSLB) WITH BIOPOLYMER SCAFFOLD AS CYTOSKELETON AND USE THEREOF,” filed Apr. 14, 2021 and U.S. Provisional Patent Application No. 63/174,148, entitled “HIGHLY STABLE LIPID BILAYER (HSLB) WITH BIOPOLYMER SCAFFOLD AS CYTOSKELETON AND USE THEREOF,” filed Apr. 13, 2021. The entire contents and disclosures of these patent applications are incorporated herein by reference in their entirety.

### STATEMENT REGARDING FEDERALLY SPONSORED RESEARCH OR DEVELOPMENT

This invention was made with the United States government support under Grant No. 1553330 awarded by National Science Foundation. The government has certain rights in the invention.

### THE NAMES OF THE PARTIES TO A JOINT RESEARCH AGREEMENT

No joint research agreement.

### BACKGROUND OF THE INVENTION

### Field of the Invention

The present disclosure relates to a device for and a method of interfacial electrofabrication of lipid bilayer supported by freestanding biopolymer membrane. The present disclosure also relates to the lipid bilayer supported by freestanding biopolymer membrane fabricated using the method provided in the present disclosure, which optionally incorporates proteins and other non-lipid molecules. At last, the present disclosure also relates to applications of the presently disclosed the lipid bilayer.

### Background Art

Lipid bilayer (LB) is a thin polar membrane made of two layers of lipid molecules that forms a continuous barrier around all cells. This film of about 50 Å thick with hydrophobic core and hydrophilic surfaces typically delimits the environments that serve as the margin between life and death for individual cells¹. Pure lipid bilayers are fragile² and are so thin that they are invisible under a traditional microscope. LB of natural biological membrane sits on the cytoskeleton layer, an intracellular matrix further supports the cell shape and functions³. Biological membranes constituents such as integral membrane proteins^{4,5} occupy around 50% of the membrane volume and further strengthen LB, as well as transport small molecules and involve in many intra- and inter-cellular signaling processes⁶. As such, natural LB is stable, fluidic and essential for cellular functions. Significantly, ion channels and molecular receptors of membrane proteins on LB are the main targets of fundamental research and pharmaceutical drugs⁷⁻⁹.

Efficient screening of the functions of membrane proteins against physical and chemical factors demands a reproducible and cost-effective method for generating LB and for measuring electrical currents through channels and pores inserted in them^{10,11}, elucidating fundamental cellular mechanisms¹² and identifying useful targets for pharmaceutical drugs⁷⁻⁹. The widely used patch clamp analysis enables recordings of electric currents through protein channels in their natural cellular environment or in artificial lipid vesicles. However, a classical patch clamp demands skilled manual operation, thereby limiting its throughput in routine drug screening assays¹¹.

Model LBs have been long used to study membrane-associated proteins that are involved in ion channel transport^{7-9,13}, membrane fusion^{3,14} and in regulation of signaling pathways^{15,16}. There are three classical model LB systems: suspended black lipid membrane (BLM), supported lipid bilayer (SLB), and tethered bilayer lipid membrane (t-BLM). Conventionally, BLM is either manually painted or constructed using monolayer opposition technique across small orifices, followed by incorporation of membrane proteins. Although a wide range of biological studies have been performed with BLMs over the past half century¹⁷⁻¹⁹, the suspended LBs often collapse within hours². SLB on solid surfaces significantly increase the stability. However, only one surface of SLB is exposed to free solution, sealing between solid surface and solution remains incomplete, and proteins are difficult to incorporate and prone to lose functions and mobility due to interaction with the solid substrate^{20,21}. For t-BLM, interaction with the solid surface is minimal and incorporation of ion channels is allowed²²⁻²⁴. However, fluidic access to t-BLM is still limited and the incomplete sealing issue is yet to be resolved.

In brief, fundamental biological studies and drug screenings depending on model LBs can enjoy a big boost if there is a LB system that is mechanically stable, allows for rapid bathing solution exchange and electrical measurement from both sides of LB, and has the potential to scale up and automate the LB formation and the subsequent analysis.

Microfluidics has emerged in the last two decades as a versatile miniaturization platform providing many advantages including portability, shorter analysis time, low sample and reagent consumption, small physical and economic footprint, parallelization and high throughput experimentation^{25,26}. Over the last decade, a variety of microfluidic systems have been developed to improve the stability and automate the manipulation of BLMs across micro- or nanopores²⁷⁻³⁰, SLB on flat surfaces by hydrogel^{21,31-33}, or the more recently developed droplet interface bilayer (DIB) between a pair of aqueous droplets in oil^{2,34-36}. Similar to the conventional BLM, freestanding BLM in microfluidics is accessible from both sides of the bilayer, but still collapse within hours due to the lack of both a supporting cytoskeleton layer that is present in live cells and the membrane constituents such as transmembrane proteins that further stabilize LB³⁷. The SLBs on solid or gel substrates generally have improved stability lasting for days. However, protein incorporation into the supported LBs remains difficult, and only one liquid compartment can be readily accessed^{21,31-33}.

Overall, LB systems in microfluidics offer enhanced functionalities and improved manipulation with respect to macroscopic laboratory setups for BLM-based applications. Nevertheless, the short lifetime of BLMs and the difficulty of protein incorporation together with limited access to SLBs still restrict the types of measurements and experiments that can be made with the current model LBs. Some researchers claim that the planar lipid bilayer technique is arguably the most technically difficult and time-consuming method in the field³⁸.

The more recently developed and the most promising DIB systems take an alternative path based on the micro-manipulation of emulsions by bringing the lipid monolayer-coated interfaces of two aqueous droplets into contact in oil^{2,34-36}. Droplet microfluidics technique has been successfully used for high throughput screenings and is an ideal tool for reproducible formation. Building upon the unique features of droplet microfluidics, these DIB systems have critical advantages such as exceptional stability for long-term measurements, micro liter consumption of samples, and the possibility of automated serial formation of LBs³¹.

Considerable effort is still needed to improve the efficiency and reliability of these systems. For example, solutions in the droplets cannot be replaced after the LB is formed without replacing the entire droplets thus disrupting the originally formed LB. Proteins and analyzing chemicals such as protein inhibitors need to be preloaded in the solution for droplets formation, which might compromise protein activities. The present of oil inside the LB is not dispensable and further compromise the protein functionalities and measurements. Finally, the DIB system without a cytoskeleton layer remains distant from a close replication of natural biological membranes.

### BRIEF DESCRIPTION OF THE DRAWINGS

These and other aspects and advantages will be described in detail with reference to the accompanying drawings, in which:

FIG. 1 is a schematic illustration showing HSLB on freestanding membrane according to an embodiment of the present disclosure.

FIG. 2 is a schematic illustration showing experimental setup and device dimensions of air bubble-initiated biofabrication according to an embodiment of the present disclosure.

FIG. 3 is a photo showing the one-step membrane formation from air-bubble trapping, dissipation, PECM formation and growth of chitosan layers according to an embodiment of the present disclosure.

FIG. 4 is an illustration showing the screening of SAR-COV-2 RNA using the lipid bilayer in the present disclosure according to an exemplary embodiment of the present disclosure.

is a schematic illustration showing the experimental setup for mechanical characterization of the biopolymer membrane with ideal gas law according to an embodiment of the present disclosure.

FIG. 5 a schematic illustration showing the structure of CAPE and DPhPC according to an exemplary embodiment of the present disclosure.

### DETAILED DESCRIPTION OF THE INVENTION

### Definitions of the Invention

When the following phrases are used substantively herein, the accompanying definitions apply. These phrases and definitions are presented without prejudice, and, consistent with the application, the right to redefine these phrases via amendment during the prosecution of this application or any application claiming priority hereto is reserved. For the purpose of interpreting a claim of any patent that claims priority hereto, each definition in that patent functions as a clear and unambiguous disavowal of the subject matter outside of that definition.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

For purposes of this present application, the terms “consisting essentially of” are to be defined according to MPEP 2111.03 Section II.

For purposes of this present application, the terms “partially tethered” are specifically defined as “1% to 98% attachment.” It should be noted that 100% attachment or support of the lipid bilayer to the freestanding biopolymer hydrogel scaffold would make the artificial cell membrane too rigid and cannot perform the expected function of a natural cell membrane.

For purposes of this present application, the term “cytoskeleton-like” is specifically defined as “an artificial biological or biopolymer structure that performs and functions as a natural cytoskeleton.”

For purposes of this present application, the terms “highly stable (in highly stable lipid bilayer)” are specifically defined as “the lipid bilayer that can maintain its functionalities and appendants for more than 4 hours.”

For purposes of this present application, the term “freestanding” is specifically defined as “the structural integrity of a membrane or cellular membrane with support or attachment only at the peripherals of the membrane.”

For purposes of this present application, the terms “flow assembly” refer to a broad range of industrial processes of forming an element by single or multiple flow of fluids.

For purposes of this present application, the terms “electrodeposition” and “electrofabrication” are used interchangeably. These terms refer to a broad range of industrial processes that assembles solid materials from molecules, ions or complexes in a solution which includes electrocoating, e-coating, cathodic electrodeposition, anodic electrodeposition and electrophoretic coating, or electrophoretic painting.

It is a conventional process of coating a thin layer of materials on conducting electrode surfaces to modify its surface properties by passing a current through an electrochemical cell from an external source. It is a versatile technique for the preparation of thin films of metals, metallic alloys, and compounds, the electrodeposited materials grow from the conductive substrate outward, and the geometry of the growth can be controlled using an insulating mask (so-called through-mask electrodeposition). However, the conventional electrodeposition has several limitations, among which the material deposition only happens on the conductive substrate, and the conductive substrate is normally part of the final product.

For purposes of this present application, the term “PECM” refers to “polyelectrolyte complex membrane.”

Descriptions of the Invention

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

The proposed HSLB system will address the notorious limitations of conventional model LB system that is crucial for fundamental biology studies involved in ion transport, membrane fusion, and regulation of signaling pathways. The research will provide a game-changer platform for studying fundamental membrane biology and for identifying membrane-associated drug targets

The HSLB was fabricated on a freestanding, semi-permeable and mechanically robust biopolymer membrane.

The supporting membrane acts as a model cytoskeleton layer of LB with high stability that presents in the natural cell membranes.

Compared to conventional model LBs, the HSLB system will provide long-term stability, accurate reproduction of cell membranes and scale-up capability, as well as enabling simultaneous fluidic, electrical and optical measurements and manipulations to study the transport activities through ion channels and the ligand-receptor interactions on cell membranes.

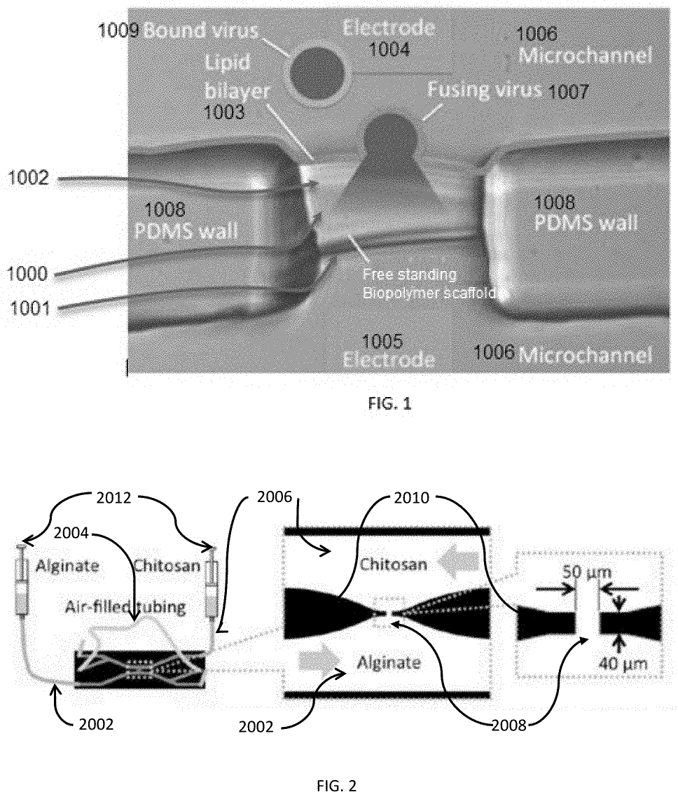

In FIG. 1, an aperture 1000 is defined by two polydimethylsiloxane (PDMS) walls 1008. A freestanding biopolymer scaffold made by chitosan membrane 1001 and alginate membrane 1002 is formed in the aperture 1000 as a biopolymer scaffold working as the cytoskeleton across the aperture. The freestanding biopolymer scaffold is formed by using spontaneous interaction between positively charged chitosan and negatively charged alginate to form a polyelectrolyte complex membrane (PECM), followed by interfacial electrofabrication of chitosan membrane onto PECM. Using the carboxyl chemistry on alginate surface (or amine chemistry on chitosan surface). The aperture diameter can be of any size, preferably 25 μm to 150 μm, and further preferably 50 μm as shown in FIG. 2. The thickness of the polydimethylsiloxane (PDMS) walls can be of any size, preferably 25 μm to 150 μm, and further preferably 40 μm as shown in FIG. 2.

The lipid bilayer 1003 is partially tethered to this freestanding biopolymer scaffold using a mixture of lipid solution (20% CAPE and 80% DPhPC for the demonstration test, other ratio to test further). The lipid bilayer 1003 forms on one surface of the biopolymer membrane. A pair of electrodes (1004 and 1005) are located on either side of the microchannel 1006.

In FIG. 1, the HSLB system is used to illustrate and study the virus-cell membrane fusion phenomena, in which a fusing virus 1007 and a bound virus 1009 are also shown in FIG. 1.

In one embodiment, the basic and novel characteristics actually are the HSLB or the artificial cell membrane consisting essentially of: a freestanding biopolymer hydrogel scaffold; a lipid bilayer; and wherein the lipid bilayer is partially tethered to the freestanding biopolymer hydrogel scaffold, and wherein the freestanding biopolymer hydrogel scaffold allows fluidic, optical and electrical access to the lipid bilayer from both sides of the lipid bilayer.

In one embodiment, the basic and novel characteristics actually are HSLB or the artificial cell membrane consisting essentially of: a freestanding biopolymer hydrogel scaffold; a lipid bilayer; and wherein the lipid bilayer is partially tethered to the freestanding biopolymer hydrogel scaffold.

In one embodiment, the HSLB platform allows for unrestricted fluidic, optical and electrical accesses to both sides of HSLB.

In one embodiment, the disclosed highly stable lipid bilayer (HSLB) system is configured to provide simultaneous fluidic, electrical and optical measurements and manipulations to the study of transport activities through ion channels and the ligand-receptor interactions on cell membranes which overcome the drawbacks of LBs in the prior arts.

In one embodiment, the vertical placement of HSLB would enable direct imaging of the spatiotemporal transport of small molecules and ions from one side of HSLB to the other side, which is impossible with the surface-supported LBs disclosed in prior arts.

In one embodiment, the HSLB platform can be scaled up and automated to fabricate thousands of HSLB within a single miniature device, which can facilitate high throughput screening applications.

### Air Bubble-Initiated Biofabrication of Biopolymer Membranes

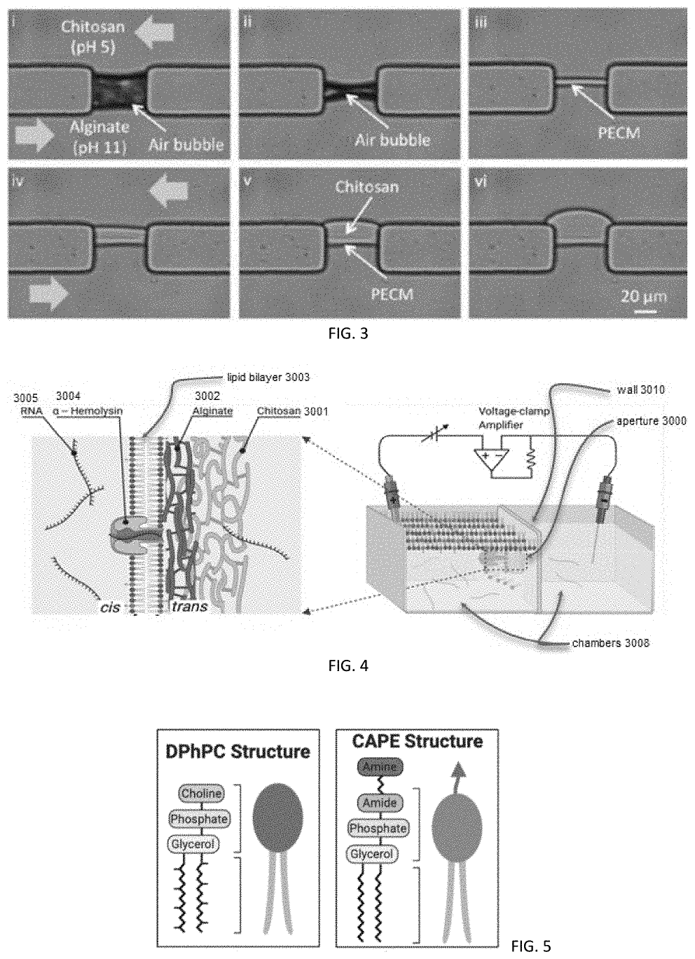

As shown in FIG. 2, an air bubble-initiated biofabrication of biopolymer membranes was initiated with intentionally entrained air bubbles within specially designed apertures in PDMS microchannels (b-i) followed by gradual dissipation of the air bubble through the gas permeable PDMS channel (b-ii)⁶⁷. FIG. 2 shows an air-filled connection tubing 2004 to balance the pressure and metal plugs 2012 to seal the outputs, the aqueous solutions, namely chitosan 2006 and alginate 2002 (chitosan of pH 5 and alginate of pH 11) separated on either side of the microchannel 2008 and PDMS walls 2010 by bubbles (FIG. 3) came in contact and formed a thin layer of polyelectrolyte complex membrane (PECM) (b-iii). FIG. 3. The chamber containing alginate is the cis (lower) chamber, while the chamber containing chitosan is the trans (upper) chamber.

During the biofabrication of the membranes, the membrane strength and potentially the permeability could be finely tuned by varying the flow rate, concentration and ionic strength of the chitosan solution, and the pH of alginate solution. The pH of chitosan is as low as 5, while the pH of alginate is as high as 11.

Due to the permeability of PECM to hydroxyl ions, chitosan molecules formed additional chitosan layers directly adjacent to the PECM (b-iv). The process is simple and requires no photo-initiator or other reagents such as those that might require removal after use. The thickness of the chitosan membranes was controlled by time and the imposed pH gradient (b-v). Portion vi of FIG. 3 Once the desired membrane thickness is reached within 3-5 minutes (b-vi), the process was stopped by withdrawing the chitosan solution and rinsing the channel with DI water. The fabrication process and results are depicted in FIG. 2(b).

In one embodiment, the walls of the microchannels are made of Teflon® film.

The process requires no sophisticated plumbing or reagents, and can be done in situ with a one-step solution introduction.

The freestanding biopolymer membranes are mechanically strong, selectively permeable to small molecules, and vertically separate the microchannels into communicating compartments.

Mechanically Strong Biopolymer Membranes Characterized with the Ideal Gas Law Principle

To characterize the mechanical strength of the fabricated biopolymer membranes, a simple and robust approach to measure the hydrostatic pressure inside microchannels⁶⁷. The approach is based on the ideal gas law principle:

PV=nRT=constant,

where P, V and n are the pressure, volume and moles, respectively, of the air plug enclosed in a syringe shown in the FIGURE, while R and T are the universal gas constant and temperature.

The ideal gas law has previously been reported for pressure measurement only in gas leak-tight silicone microchannels⁶⁸. For the first time, the ideal gas law was applied to measure pressure in gas-permeable PDMS microchannels.

### Screen SAR-COV-2 RNA

FIGS. 4 and 5 shown the highly stable lipid bilayer system in the present disclosure is used to test SARS-CoV-2 RNA.

A freestanding chitosan membrane 3001 acting as a biopolymer scaffold working as the cytoskeleton across a Teflon aperture 3000, was formed. This is done by using spontaneous interaction between positively charged chitosan and negatively charged alginate to form a polyelectrolyte complex membrane (PECM), followed by interfacial electrofabrication of chitosan membrane onto PECM. The PECM comprises of the chitosan membrane 3001 and alginate membrane 3002.

Using the carboxyl chemistry on alginate surface (or amine chemistry on chitosan surface), a lipid bilayer 3003 is partially tethered to this biopolymer scaffold using a mixture of lipid solution.

The highly stable lipid bilayer system incorporated with membrane protein 3004 (alpha-hemolysin) is used to screen SARS-CoV-2 RNA 3005.

The solutions in both chambers 3008 can be replaced with new sample that needs to be tested.

The SARS-CoV-2 RNA is translocated through the membrane protein in the lipid bilayer. Translocation of RNA through the membrane protein generates current signal that is detected by the voltage-clamp amplifier and analyzed after recording. The current signal generated during the translocation of spike mRNA.

The embedded membrane protein in the lipid bilayer system remains functional and robust after solution draining and refilling.

The lipid bilayer system with incorporated membrane protein enables the rapid testing of SARS-CoV-2 and reusability of this system by simply changing the measured sample solution with new sample solution and buffer.

The artificial cell membrane comprising: a freestanding biopolymer hydrogel scaffold; a lipid bilayer; wherein the lipid bilayer is partially tethered to the freestanding biopolymer hydrogel scaffold, and wherein the freestanding biopolymer hydrogel scaffold allows fluidic, optical and electrical access to the lipid bilayer from both sides of the lipid bilayer.

The artificial cell membrane as above, wherein the freestanding biopolymer hydrogel scaffold is at least one of: a chitosan layer, an alginate layer and a combination thereof.

The artificial cell membrane as above, wherein the freestanding biopolymer hydrogel scaffold is at least one of: an anode biopolymer electrolyte, a cathode electrolyte, and a combination thereof.

The artificial cell membrane as above, wherein the anode biopolymer electrolyte is selected from, including but not limited to, the group consisting of chitosan, poly-L-lysine (PLL), polyethylenimine (PEI), and diethylaminoethyl-dextran (DEAE-DEX) solutions; and wherein the cathode electrolyte is selected from but not limited to the group consisting of alginate, polystyrene sulfonates (PSS), and polyacrylic acid (PAA) solutions.

The artificial cell membrane as above, wherein the freestanding biopolymer hydrogel scaffold is semi-permeable, cytoskeleton-like, biopolymer scaffold.

The artificial cell membrane as above, wherein the artificial cell membrane is a highly stable lipid bilayer (HSLB) system.

The artificial cell membrane as above, wherein only one side of the lipid bilayer is partially tethered to the freestanding biopolymer hydrogel scaffold.

The artificial cell membrane as above, wherein the artificial cell membrane or the lipid bilayer is configured to incorporate other natural or artificial components that are configured to associate with a natural cell membrane.

The artificial cell membrane as above, wherein the freestanding biopolymer hydrogel scaffold is formed by interaction between positively charged chitosan and negatively charged alginate to form a polyelectrolyte complex membrane (PECM).

The artificial cell membrane as above, wherein the artificial cell membrane and the lipid bilayer is stable and functional for at least 4 hours.

The artificial cell membrane as above, wherein the artificial cell membrane will remain structurally sound even with the withdrawal and refilling of the surrounding aqueous solutions and the artificial cell membrane is configured for fast exchange of the aqueous solutions.

The artificial cell membrane consisting essentially of: a freestanding biopolymer hydrogel scaffold; a lipid bilayer; and wherein the lipid bilayer is partially tethered to the freestanding biopolymer hydrogel scaffold.

The invention including a method of forming an artificial cell membrane comprising: forming a freestanding biopolymer hydrogel scaffold; adding a lipid solution to the freestanding biopolymer hydrogel scaffold and allowing the lipid molecules to partially tether to the freestanding biopolymer hydrogel scaffold; thus, forming a lipid bilayer.

The method as above, wherein the freestanding biopolymer hydrogel scaffold is at least one of: a chitosan layer, an alginate layer and a combination thereof.

The method as above, wherein the freestanding biopolymer hydrogel scaffold is semi-permeable, cytoskeleton-like, biopolymer scaffold.

The method as above, wherein the freestanding biopolymer hydrogel scaffold is formed by interaction between positively charged chitosan and negatively charged alginate to form a polyelectrolyte complex membrane (PECM).

The method as above, wherein the lipid bilayer is formed, including but not limited to, using a mixture of lipid solution comprising charged and uncharged lipids.

The method as above, wherein the lipid bilayer is formed, including but not limited to, using a mixture of lipid or liposome solution selected from the group consisting of: caffeic acid phenethyl ester (CAPE), Diphytanoyl phosphatidylcholine lipids (DPhPC), diphytanoyl phosphoethanolamine lipids (DPhPE) and a combination thereof.

The method as above, wherein the freestanding biopolymer hydrogel scaffold is formed by interfacial electrofabrication process or flow assembly process.

The method as above, wherein other natural or artificial components that are configured to associate with a natural cell membrane can be incorporated into the artificial cell membrane or the lipid bilayer to mimic the functionalities of a natural cell membrane.

The method as above, wherein the lipid bilayer is formed by self-assembling of lipid molecules from a mixture of lipid solution.

The method as above, wherein the lipid bilayer is configured to tether on either side of the freestanding biopolymer hydrogel scaffold.

### Scale Up the Model HSLB and Make it Available for Other Users

The HSLB supported on the semi-permeable, cytoskeleton-like scaffold provides long-term stability, accurate reproduction of cell membranes, as well as enabling simultaneous fluidic, electrical and optical measurements and manipulations to study ion channel activities and ligand-receptor interactions on cell membranes. The model HSLB system is batch produced using the device in the present disclosure.

To mass produce HSLB, one key effort is to batch fabricate the freestanding biopolymer membrane. In one embodiment, the batch fabricatiom of the freestanding biopolymer membrane is achieved by improving the biopolymer membrane biofabrication process with two-layer microfluidic devices including a PDMS gas layer and PDMS fluidic layer.

The biopolymer membrane biofabrication device is a one-layer device expel air bubbles trapped in apertures out of PDMS by creating positive pressure as well as balancing the pressure with air-filled tubing connecting the fluidic outputs.

Alternatively, the biopolymer membrane biofabrication device is a two-layer device, with one-step solution introduction. withdrawing of air through an additional gas channels above the apertures creates negative pressure to suck the air bubbles out of PDMS, while the output of the fluidic channels is open to the atmosphere. In this way, the pressure balancing in the biofabrication process is individually monitored and adjusted, so that the biofabrication process with one-step solution introduction can be easily scaled up.

### REFERENCES

The following references are referred to above and are incorporated herein by reference:
1. van Meer, G., Voelker, D. R. & Feigenson, G. W. Membrane lipids: where they are and how they behave. Nature Reviews Molecular Cell Biology 9, 112-124, doi:10.1038/nrm2330 (2008).
2. Bayley, H. et al. Droplet interface bilayers. Mol. Biosyst. 4, 1191-1208, doi:10.1039/b808893d (2008).
3. Yin, H. L. & Janmey, P. A. Phosphoinositide regulation of the actin cytoskeleton. Annu. Rev. Physiol. 65, 761-789, doi:10.1146/annurev.physio1.65.092101.142517 (2003).
4. Lee, A. G. How lipids affect the activities of integral membrane proteins. Biochimica Et Biophysica Acta-Biomembranes 1666, 62-87, doi:10.1016/j.bbamem.2004.05.012 (2004).
5. Lee, A. G. Lipid-protein interactions in biological membranes: a structural perspective. Biochimica Et Biophysica Acta-Biomembranes 1612, 1-40, doi:10.1016/s0005-2736(03)00056-7 (2003).
6. Groves, J. T. & Kuriyan, J. Molecular mechanisms in signal transduction at the membrane. Nature Structural & Molecular Biology 17, 659-665, doi:10.1038/nsmb.1844 (2010).
7 Szymanski, W., Yilmaz, D., Kocer, A. & Feringa, B. L. Bright Ion Channels and Lipid Bilayers. Accounts of Chemical Research 46, 2910-2923, doi:10.1021/ar4000357 (2013).
8. Kamkin, A. G., Kiseleva, I. S. & Yarigin, V. N. Mechanosensitive ion channels. Usp. Fiziol. Nauk 33, 3-37 (2002).
9. Reiss, P. & Koert, U. Ion-Channels: Goals for Function-Oriented Synthesis. Accounts of Chemical Research 46, 2773-2780, doi:10.1021/ar400007w (2013).
10. Dunlop, J., Bowlby, M., Peri, R., Vasilyev, D. & Arias, R. High-throughput electrophysiology: an emerging paradigm for ion-channel screening and physiology. Nature Reviews Drug Discovery 7, 358-368, doi:10.1038/nrd2552 (2008).
11. Czekalska, M. A. et al. A droplet microfluidic system for sequential generation of lipid bilayers and transmembrane electrical recordings. Lab on a Chip 15, 541-548, doi:10.1039/c41c00985a (2015).
12. Simons, K. & Vaz, W. L. C. Model systems, lipid rafts, and cell membranes. Annu. Rev. Biophys. Biomol. Struct. 33, 269-295, doi:10.1146/annurev.biophys.32.110601.141803 (2004).
13. Levitan, I., Fang, Y., Rosenhouse-Dantsker, A. & Romanenko, V. in Cholesterol Binding and Cholesterol Transport Proteins: Structure and Function in Health and Disease Vol. 51 Subcellular Biochemistry (ed J. R. Harris) 509-549 (2010).
14. Jahn, R., Lang, T. & Sudhof, T. C. Membrane fusion. Cell 112, 519-533, doi:10.1016/s0092-8674(03)00112-0 (2003).
15. Hamill, 0. P. & Martinac, B. Molecular basis of mechanotransduction in living cells. Physiol. Rev. 81, 685-740 (2001).
16. Mannock, D. A., Lewis, R. N. A. H., McMullen, T. P. W. & McElhaney, R. N. The effect of variations in phospholipid and sterol structure on the nature of lipid-sterol interactions in lipid bilayer model membranes. Chem. Phys. Lipids 163, 403-448, doi:10.1016/j.chemphyslip.2010.03.011 (2010).
17. Simons, K. & Ikonen, E. Functional rafts in cell membranes. Nature 387, 569-572, doi: 10.1038/42408(1997).
18. Nikaido, H. Molecular basis of bacterial outer membrane permeability revisited. Microbiol. Mol. Biol. Rev. 67, 593-+, doi:10.1128/mmbr.67.4.593-656.2003 (2003).
19. Lingwood, D. & Simons, K. Lipid Rafts As a Membrane-Organizing Principle. Science 327, 46-50, doi:10.1126/science.1174621 (2010).
20. Tamm, L. K. & McConnell, H. M. Supported phospholipid bilayers. Biophys. J. 47, 105-113, doi:10.1016/S0006-3495(85)83882-0 (1985).
21. Castellana, E. T. & Cremer, P. S. Solid supported lipid bilayers: From biophysical studies to sensor design. Surface Science Reports 61, 429-444, doi:10.1016/j.surfrep.2006.06.001 (2006).
22. Wagner, M. L. & Tamm, L. K. Tethered polymer-supported planar lipid bilayers for reconstitution of integral membrane proteins: Silane-polyethyleneglycol-lipid as a cushion and covalent linker. Biophys. J. 79, 1400-1414 (2000).
23. Raguse, B. et al. Tethered lipid bilayer membranes: Formation and ionic reservoir characterization. Langmuir 14, 648-659, doi:10.1021/1a9711239 (1998).
24. Graneli, A., Yeykal, C. C., Prasad, T. K. & Greene, E. C. Organized arrays of individual DNA molecules tethered to supported lipid bilayers. Langmuir 22, 292-299, doi:10.1021/1a051944a (2006).
25. Whitesides, G. M. The origins and the future of microfluidics. Nature 442,368-373, doi:10.1038/nature05058 (2006).
26. Stone, H. A., Stroock, A. D. & Ajdari, A. Engineering flows in small devices: Microfluidics toward a lab-on-a-chip. Annual Review of Fluid Mechanics 36, 381-411, doi:10.1146/annurev.fluid.36.050802.122124 (2004).
27. Kawano, R., Osaki, T., Sasaki, H. & Takeuchi, S. A Polymer-Based Nanopore-Integrated Microfluidic Device for Generating Stable Bilayer Lipid Membranes. Small 6, 2100-2104, doi:10.1002/sm11.201000997 (2010).
28. Zagnoni, M., Sandison, M. E. & Morgan, H. Microfluidic array platform for simultaneous lipid bilayer membrane formation. Biosens. Bioelectron. 24, 1235-1240, doi:10.1016/j.bios.2008.07.022 (2009).
29. Suzuki, H., Tabata, K. V., Noji, H. & Takeuchi, S. Highly reproducible method of planar lipid bilayer reconstitution in polymethyl methacrylate microfluidic chip. Langmuir 22, 1937-1942, doi:10.1021/1a052534p (2006).
30. Bally, M. et al. Liposome and Lipid Bilayer Arrays Towards Biosensing Applications. Small 6, 2481-2497, doi:10.1002/sm11.201000644 (2010).
31. Moran-Mirabal, J. M. et al. Micrometer-sized supported lipid bilayer arrays for bacterial toxin binding studies through total internal reflection fluorescence microscopy. Biophys. J. 89, 296-305, doi:10.1529/biophysj.104.054346 (2005).
32. Kim, P. et al. Soft lithographic patterning of supported lipid bilayers onto a surface and inside microfluidic channels. Lab on a Chip 6, 54-59, doi:10.1039/b512593f (2006).
33. Burridge, K. A., Figa, M. A. & Wong, J. Y. Patterning adjacent supported lipid bilayers of desired composition to investigate receptor-ligand binding under shear flow. Langmuir 20, 10252-10259, doi:10.1021/1a0489099 (2004).
34. Stanley, C. E. et al. A microfluidic approach for high-throughput droplet interface bilayer (DIB) formation. Chemical Communications 46, 1620-1622, doi:10.1039/b924897h (2010).
35. Heron, A. J., Thompson, J. R., Mason, A. E. & Wallace, M. I. Direct detection of membrane channels from gels using water-in-oil droplet bilayers. J. Am. Chem. Soc. 129, 16042-16047, doi:10.1021/ja075715h (2007).
36. Funakoshi, K., Suzuki, H. & Takeuchi, S. Lipid bilayer formation by contacting monolayers in a microfluidic device for membrane protein analysis. Anal. Chem. 78, 8169-8174, doi:10.1021/ac0613479 (2006).
37. Malmstadt, N., Nash, M. A., Purnell, R. F. & Schmidt, J. J. Automated formation of lipid-bilayer membranes in a microfluidic device. Nano Letters 6, 1961-1965, doi:10.1021/n10611034 (2006).
38. Costa, J. A., Nguyen, D. A., Leal-Pinto, E., Gordon, R. E. & Hanss, B. Wicking: A Rapid Method for Manually Inserting Ion Channels into Planar Lipid Bilayers. PLoS One 8, e60836, doi:10.1371/journal.pone.0060836 (2013).
39. Ota, S., Suzuki, H. & Takeuchi, S. Microfluidic lipid membrane formation on microchamber arrays. Lab on a Chip 11, 2485-2487, doi:10.1039/c1lc20334g (2011).
40. Zagnoni, M., Sandison, M. E. & Morgan, H. Microfluidic array platform for simultaneous lipid bilayer membrane formation. Biosens. Bioelectron. 24, 1235-1240, doi:10.1016/j.bios.2008.07.022 (2009).
41. Holden, M. A., Needham, D. & Bayley, H. Functional bionetworks from nanoliter water droplets. J. Am. Chem. Soc. 129, 8650-8655, doi:10.1021/ja072292a (2007).
42. Zhou, X., Moran-Mirabal, J. M., Craighead, H. G. & McEuen, P. L. Supported lipid bilayer/carbon nanotube hybrids. Nat Nano 2, 185-190, doi: 10.1038/nnano.2007.34_S1.html (2007).
43. Lu, B., Kocharyan, G. & Schmidt, J. J. Lipid bilayer arrays: Cyclically formed and measured. Biotechnology Journal 9, 446-451, doi:10.1002/biot.201300271 (2014).
44. Robison, A. D., Huang, D., Jung, H. & Cremer, P. S. Fluorescence modulation sensing of positively and negatively charged proteins on lipid bilayers. Biointerphases 8, 1, doi: 10.1186/1559-4106-8-1 (2013).
45. Boreyko, J. B., Mruetusatorn, P., Sarles, S. A., Retterer, S. T. & Collier, C. P. Evaporation-Induced Buckling and Fission of Microscale Droplet Interface Bilayers. J. Am. Chem. Soc. 135, 5545-5548, doi:10.1021/ja4019435 (2013).
46. Phillips, K. S. & Cheng, Q. Microfluidic immunoassay for bacterial toxins with supported phospholipid bilayer membranes on poly(dimethylsiloxane). Anal. Chem. 77, 327-334, doi:10.1021/ac049356+(2005).
47. Dong, Y., Phillips, K. S. & Cheng, Q. Immunosensing of Staphylococcus enterotoxin B (SEB) in milk with PDMS microfluidic systems using reinforced supported bilayer membranes (r-SBMs). Lab on a Chip 6, 675-681, doi:10.1039/b514902a (2006).
48. Stachowiak, J. C. et al. Unilamellar vesicle formation and encapsulation by microfluidic jetting. Proc. Natl. Acad. Sci. U.S.A 105, 4697-4702, doi:10.1073/pnas.0710875105 (2008).
49. Jahn, A. et al. Microfluidic Mixing and the Formation of Nanoscale Lipid Vesicles. Acs Nano 4,2077-2087, doi:10.1021/nn901676x (2010).
50. Richmond, D. L. et al. Forming giant vesicles with controlled membrane composition, asymmetry, and contents. Proc. Natl. Acad. Sci. U.S.A 108, 9431-9436, doi:10.1073/pnas.1016410108 (2011).
51. Matosevic, S. & Paegel, B. M. Stepwise Synthesis of Giant Unilamellar Vesicles on a Microfluidic Assembly Line. J. Am. Chem. Soc. 133, 2798-2800, doi:10.1021/ja109137s (2011).
52. Matosevic, S. & Paegel, B. M. Layer-by-layer cell membrane assembly. Nature Chemistry 5,958-963, doi:10.1038/nchem.1765 (2013).
53. Sarles, S. A. & Leo, D. J. Regulated Attachment Method for Reconstituting Lipid Bilayers of Prescribed Size within Flexible Substrates. Anal. Chem. 82, 959-966, doi:10.1021/ac902555z (2010).
54. Sarles, S. A. & Leo, D. J. Physical encapsulation of droplet interface bilayers for durable, portable biomolecular networks. Lab on a Chip 10, 710-717, doi:10.1039/b916736f (2010).
55. Shao, C., Kendall, E. L. & DeVoe, D. L. Electro-optical BLM chips enabling dynamic imaging of ordered lipid domains. Lab on a Chip 12, 3142-3149, doi:10.1039/c21c40077d (2012).
56. Luo, X.L., Buckhout-White, S., Bentley, W. E. & Rubloff, G. W. Biofabrication of chitosan-silver composite SERS substrates enabling quantification of adenine by a spectroscopic shift. Biofabrication 3, 034108, doi: 10.1088/1758-5082/3/3/034108 (2011).
57. Fernandes, R. et al. Biological nanofactories facilitates patially selective capture and manipulation of quorum sensing bacteria in a bioMEMS device. Lab on a Chip 10, 1128-1134, doi:10.1039/b926846d (2010).
58. Luo, X. et al. Programmable assembly of a metabolic pathway enzyme in a pre-packaged reusable bioMEMS device. Lab on a Chip 8, 420-430, doi:10.1039/b713756g (2008).
59. Luo, X. et al. Design optimization for bioMEMS studies of enzyme-controlled metabolic pathways. Biomedical Microdevices 10, 899-908, doi: 10.1007/s10544-008-9204-5 (2008).
60. Cheng, Y. et al. Biocompatible multi-address 3D cell assembly in microfluidic devices usings patially programmable gel formation. Lab on a Chip 11, 2316-2318, doi:10.1039/c11c20306a (2011).
61. Cheng, Y. et al. In situ quantitative visualization and characterization of chitosan electrodeposition with paired sidewall electrodes. Soft Matter 6, 3177-3183, doi:10.1039/c0sm00124d (2010).
62. Betz, J. F. et al. Optically clear alginate hydrogels for spatially controlled cell entrapment and culture at microfluidic electrode surfaces. Lab on a Chip 13, 1854-1858, doi:10.1039/c31c50079a (2013).
63. Luo, X. et al. Biofabrication of stratified biofilm mimics for observation and control of bacterial signaling. Biomaterials 33, 5136-5143, doi:10.1016/j.biomaterials.2012.03.037 (2012).
64. Luo, X. L. et al. In situ generation of pH gradients in microfluidic devices for biofabrication of freestanding, semi-permeable chitosan membranes. Lab on a Chip 10, 59-65, doi:10.1039/b916548g (2010).
65. Luo, X. et al. Distal modulation of bacterial cell-cell signalling in a synthetic ecosystem using partitioned microfluidics. Lab on a Chip 15, 1842-1851, doi:10.1039/c51c00107b (2015).
66. Lewandowski, A. T. et al. Protein assembly onto patterned microfabricated devices through enzymatic activation of fusion pro-tag. Biotechnol. Bioeng. 99, 499-507 (2008).
67. Luo, X., Wu, H.-C., Betz, J., Rubloff, G. W. & Bentley, W. E. Air bubble-initiated biofabrication of freestanding, semi-permeable biopolymer membranes in PDMS microfluidics. Biochemical Engineering Journal 89, 2-9, doi:10.1016/j.bej.2013.12.13 (2014).
68. Srivastava, N. & Burns, M. A. Microfluidic pressure sensing using trapped air compression. Lab on a Chip 7, 633-637, doi:10.1039/b617067f (2007).
69. Guo, M. et al. Geminal dihalogen isosteric replacement in hydrated AI-2 affords potent quorum sensing modulators. Chemical Communications 51, 2617-2620, doi:10.1039/c4cc09361e (2015).
70. Lentini, R. et al. Integrating artificial with natural cells to translate chemical messages that direct E. coli behaviour. Nature Communications 5, 4012, doi:10.1038/ncomms5012 (2014).
71. Guo, M. et al. 3-Aminooxazolidinone AHL analogs as hydrolytically-stable quorum sensing agonists in Gram-negative bacteria. Medchemcomm 6, 1086-1092, doi:10.1039/c5md00015g (2015).
72. Gordonov, T. et al. Electronic modulation of biochemical signal generation. Nature Nanotechnology 9, 605-610, doi:10.1038/nnano.2014.151 (2014).
73. Servinsky, M. D. et al. Directed assembly of a bacterial quorum. ISME J, doi:10.1038/ismej.2015.89 (2015).
74. Zargar, A. et al. Bacterial Secretions of Nonpathogenic Escherichia coli Elicit Inflammatory Pathways: a Closer Investigation of Interkingdom Signaling. Mbio 6, e00025-15, doi:10.1128/mBio.00025-15 (2015).
75. Zargar, A. et al. Rational design of ‘controller cells’ to manipulate protein and phenotype expression. Metabolic Engineering 30, 61-68, doi:10.1016/j.ymben.2015.04.001 (2015).
76. Teng, W., Ban, C. & Hahn, J. H. Formation of lipid bilayer membrane in a poly(dimethylsiloxane) microchip integrated with a stacked polycarbonate membrane support and an on-site nanoinjector. Biomicrofluidics 9, 024120, doi:10.1063/1.4919066 (2015).
77. Basit, H., Gaul, V., Maher, S., Forster, R. J. & Keyes, T. E. Aqueous-filled polymer microcavity arrays: versatile & stable lipid bilayer platforms offering high lateral mobility to incorporated membrane proteins. Analyst 140, 3012-3018, doi:10.1039/c4an02317j (2015).
78. Ryu, H. et al. Automated Lipid Membrane Formation Using a Polydimethylsiloxane Film for Ion Channel Measurements. Anal. Chem. 86, 8910-8915, doi:10.1021/ac501397t (2014).
79. Saranathan, V. et al. Structural Diversity of Arthropod Biophotonic Nanostructures Spans Amphiphilic Phase-Space. Nano Letters 15, 3735-3742, doi:10.1021/acs.nanolett.5b00201 (2015).
80. Zhang, X., Tanner, P., Graff, A., Palivan, C. G. & Meier, W. Mimicking the cell membrane with block copolymer membranes. Journal of Polymer Science Part a-Polymer Chemistry 50, 2293-2318, doi:10.1002/pola.26000 (2012).
81. Schulz, M. et al. Hybridlipid/polymer giant unilamellar vesicles: effects of incorporated biocompatible PIB-PEO block copolymers on vesicle properties. Soft Matter 7, 8100-8110, doi:10.1039/c1sm05725a (2011).
82. Rangelov, S., Almgren, M., Edwards, K. & Tsvetanov, C. Formation of normalandreversebilayer structures by self-assembly of nonionic block copolymers bearing lipid-mimetic units. Journal of Physical Chemistry B 108, 7542-7552, doi:10.1021/jp0304576 (2004).
83. Zagnoni, M. Miniaturised technologies for the development of artificial lipid bilayer systems. Lab on a Chip 12, 1026-1039, doi:10.1039/c21c20991h (2012).
84. Olapinski, M. et al. Detection of lipid bilayer and peptide pore formation at gigahertz frequencies. Applied Physics Letters 88, 013902, doi:10.1063/1.2159571 (2006).
85. Olapinski, M. et al. Probing lipid membranes and ion channels with high frequency spectroscopy. (2004).
86. Maki, Y. et al. Universality and specificity in molecular orientation in an isotropic gels prepared by diffusion method. Carbohydrate Polymers 108, 118-126, doi:10.1016/j.carbpol.2014.03.012 (2014).
87. Fernandez, R., Ocando, C., Fernandes, S. C. M., Eceiza, A. & Tercjak, A. Optically Active Multilayer Films Based on Chitosan and an Azopolymer. Biomacromolecules 15, 1399-1407, doi:10.1021/bm 500014r (2014).
88. Lilly, J. L., Romero, G., Xu, W., Shin, H. Y. & Berron, B. J. Characterization of Molecular Transportin Ultrathin Hydrogel Coatings for Cellular Immunoprotection. Biomacromolecules 16, 541-549, doi: 10.1021/bm 501594x (2015).
89. Lundbaek, J. A., Collingwood, S. A., Ingolfsson, H. I., Kapoor, R. & Andersen, 0. S. Lipid bilayer regulation of membrane protein function: gramicidin channels as molecular force probes. Journal of the Royal Society Interface 7, 373-395, doi:10.1098/rsif.2009.0443 (2010).
90. Prenner, E. J., Lewis, R. & McElhaney, R. N. The interaction of the antimicrobial peptide Gramicidin S with lipid bilayer model and biological membranes. Biochimica Et Biophysica Acta-Biomembranes 1462, 201-221, doi: 10.1016/s0005-2736(99)00207-2 (1999).
91. Woolley, G. A. & Wallace, B. A. MODEL ION CHANNELS-GRAMICIDIN AND ALAMETHICIN. J. Membr. Biol. 129, 109-136 (1992).
92. Eisele, F., Kuhlmann, J. & Waldmann, H. Synthesis and membrane binding properties of a lipopeptide fragment from influenza virus A hemagglutinin. Chemistry-a European Journal 8, 3362-3376, doi:10.1002/1521-3765(20020802)8:15<3362::aid-chem3362>3.0.co; 2-0 (2002).
93. Nunes-Correia, I., Ramalho-Santos, J., Nir, S. & deLima, M. C. P. Interactions of influenza virus with cultured cells: Detailed kinetic modeling of binding and endocytosis. Biochemistry (Mosc). 38, 1095-1101, doi:10.1021/bi9812524 (1999).
94. Harrison, S. C. Viral membrane fusion. Virology 479,498-507, doi:10.1016/j.viro1.2015.03.043(2015).
95. Sollenberger, D. J. & Singh, M. P. in Agent-Oriented Software Engineering X Vol. 6038 Lecture Notes in Computer Science (eds M. P. Gleizes & J. J. GomezSanz) 97-109 (2011).
96. Ghosh, S. & Ieee; Ieee, I. in 28th Annual Frontiers in Education Conference-Conference Proceedings, Vols 1-3 Proceedings-Frontiers in Education Conference 322-327 (1998).
97. deJong, J., Ankone, B., Lammertink, R. G. H. & Wessling, M. New replication technique for the fabrication of thin polymeric microfluidic devices with tunable porosity. Lab on a Chip 5, 1240-1247, doi:10.1039/b509280a (2005).
98. Delille, R., Urdaneta, M. G., Moseley, S. J. & Smela, E. Benchtop polymer MEMS. Journal of Microelectromechanical Systems 15, 1108-1120, doi:10.1109/jmems.2006.882610 (2006).
99. Jara, C. A., Candelas, F. A., Puente, S. T. & Torres, F. Hands-on experiences of undergraduate students in Automatics and Robotics using a virtual and remote laboratory. Computers & Education 57, 2451-2461, doi:10.1016/j.compedu.2011.07.003 (2011).
100. Radharamanan, R. & Jenkins, H. E. Laboratory learning modules on CAD/CAM and robotics in engineering education. International Journal of Innovative Computing Information and Control 4, 433-443 (2008).
101. Vallim, M. B. R., Farines, J. M. & Cury, J. E. R. Practicing engineering in a freshman introductory course. Ieee Transactions on Education 49, 74-79, doi:10.1109/te.2005.856157 (2006).
102. Jacobson, M. L., Said, R. a. A. & Rehman, H.-U. Introducing design skills at the freshman level: Structured design experience. Ieee Transactions on Education 49, 247-253, doi:10.1109/te.2006.872403 (2006).
103. Byars-Winston, A., Estrada, Y., Howard, C., Davis, D. & Zalapa, J. Influence of Social Cognitive and Ethnic Variables on Academic Goals of Underrepresented Students in Science and Engineering: A Multiple-Groups Analysis. Journal of Counseling Psychology 57, 205-218, doi:10.1037/a0018608 (2010).
104. Balster, N., Pfund, C., Rediske, R. & Branchaw, J. Entering Research: A Course That Creates Community and Structure for Beginning Undergraduate Researchers in the STEM Disciplines. Cbe-Life Sciences Education 9, 108-118, doi:10.1187/cbe.09-10-0073 (2010).
105. Lewis, J. L., Menzies, H., Najera, E. I. & Page, R. N. Rethinking Trends in Minority Participation in the Sciences. Science Education 93, 961-977, doi:10.1002/sce.20338 (2009).
106. Zimmerman, J. B. & Vanegas, J. Using Sustainability education to enable the increase of diversity in science, engineering and technology-related disciplines. International Journal of Engineering Education 23, 242-253 (2007).

All documents, patents, journal articles and other materials cited in the present application are incorporated herein by reference.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

We claim:

1. An artificial cell membrane comprising:
a freestanding biopolymer hydrogel scaffold;
a lipid bilayer;
wherein the lipid bilayer is partially tethered to the freestanding biopolymer hydrogel scaffold, and
wherein the freestanding biopolymer hydrogel scaffold allows fluidic, optical and electrical access to the lipid bilayer from both sides of the lipid bilayer.

2. The artificial cell membrane as in claim 1, wherein the freestanding biopolymer hydrogel scaffold is at least one of: an anode biopolymer electrolyte, a cathode electrolyte, and a combination thereof.

3. The artificial cell membrane as in claim 2, wherein the anode biopolymer electrolyte is selected from, including but not limited to, the group consisting of chitosan, poly-L-lysine (PLL), polyethylenimine (PEI), and diethylaminoethyl-dextran (DEAE-DEX) solutions; and
wherein the cathode electrolyte is selected from but not limited to the group consisting of alginate, polystyrene sulfonates (PSS), and polyacrylic acid (PAA) solutions.

4. The artificial cell membrane as in claim 1, wherein the artificial cell membrane is a highly stable lipid bilayer (HSLB) system.

5. The artificial cell membrane as in claim 1, wherein only one side of the lipid bilayer is partially tethered to the freestanding biopolymer hydrogel scaffold.

6. The artificial cell membrane as in claim 1, wherein the artificial cell membrane or the lipid bilayer is configured to incorporate other natural or artificial components that are configured to associate with a natural cell membrane.

7. The artificial cell membrane as in claim 1, wherein the freestanding biopolymer hydrogel scaffold is formed by interaction between positively charged chitosan and negatively charged alginate to form a polyelectrolyte complex membrane (PECM).

8. The artificial cell membrane as in claim 1, wherein the artificial cell membrane and the lipid bilayer is stable and functional for at least 4 hours.

9. The artificial cell membrane as in claim 1, wherein the artificial cell membrane will remain structurally sound even with the withdrawal and refilling of the surrounding aqueous solutions and the artificial cell membrane is configured for fast exchange of the aqueous solutions.

10. An artificial cell membrane consisting essentially of:
a freestanding biopolymer hydrogel scaffold;
a lipid bilayer; and
wherein the lipid bilayer is partially tethered to the freestanding biopolymer hydrogel scaffold.

11. A method of forming an artificial cell membrane comprising:
forming a freestanding biopolymer hydrogel scaffold;
adding a lipid solution to the freestanding biopolymer hydrogel scaffold and allowing the lipid molecules to partially tether to the freestanding biopolymer hydrogel scaffold;
thus, forming a lipid bilayer.

12. The method of claim 11, wherein the freestanding biopolymer hydrogel scaffold is at least one of: an anode biopolymer electrolyte, a cathode electrolyte, and a combination thereof.

13. The method of claim 12, wherein the anode biopolymer electrolyte is selected from, including but not limited to, the group consisting of chitosan, poly-L-lysine (PLL), polyethylenimine (PEI), and diethylaminoethyl-dextran (DEAE-DEX) solutions; and
wherein the cathode electrolyte is selected from but not limited to the group consisting of alginate, polystyrene sulfonates (PSS), and polyacrylic acid (PAA) solutions.

14. The method of claim 11, wherein the freestanding biopolymer hydrogel scaffold is formed by interaction between positively charged chitosan and negatively charged alginate to form a polyelectrolyte complex membrane (PECM).

15. The method of claim 11, wherein the lipid bilayer is formed, including but not limited to, using a mixture of lipid solution comprising charged and uncharged lipids.

16. The method of claim 11, wherein the lipid bilayer is formed, including but not limited to, using a mixture of lipid or liposome solution selected from the group consisting of: caffeic acid phenethyl ester (CAPE), Diphytanoyl phosphatidylcholine lipids (DPhPC), diphytanoyl phosphoethanolamine lipids (DPhPE) and a combination thereof.

17. The method of claim 11, wherein the freestanding biopolymer hydrogel scaffold is formed by interfacial electrofabrication process or flow assembly process.

18. The method of claim 11, wherein other natural or artificial components that are configured to associate with a natural cell membrane can be incorporated into the artificial cell membrane or the lipid bilayer to mimic the functionalities of a natural cell membrane.

19. The method of claim 11, wherein the lipid bilayer is formed by self-assembling of lipid molecules from a mixture of lipid solution.

20. The method of claim 11, wherein the lipid bilayer is configured to tether on either side of the freestanding biopolymer hydrogel scaffold.
